(19) United States
(12) Patent Application Publication
(10) Pub. No.: US 2007/0196863 A1
(43) Pub. Date: Aug. 23, 2007

(54) PRION PROTEIN DETECTION

(75) Inventors: Maureen Angela DYER, Mechanicsburg, PA (US); William Paynter HANSON, Carlisle, PA (US); Thrygve Richard MEEKER, Allentown, PA (US)

Correspondence Address: MCNEES WALLACE & NURICK LLC, 100 PINE STREET, P.O. BOX 1166, HARRISBURG, PA, 17108-1166, US

(73) Assignee: HANSON TECHNOLOGIES, INC., Carlisle, PA (US)

(21) Appl. No.: 11/675,950

(22) Filed: Feb. 16, 2007

Related U.S. Application Data
(60) Provisional application No. 60/774,345, filed on Feb. 17, 2006.
(60) Provisional application No. 60/779,620, filed on Mar. 6, 2006.

(51) Int. Cl. G01N 33/53; C12M 1/34; G01J 3/00

(52) U.S. Cl. 435/7.1; 435/287.2; 356/300

(57) ABSTRACT

An example embodiment of the invention includes a method of performing an assay comprising the steps of (1) providing a multimode waveguide; (2) fixing one or more fluidic cells to the multimode waveguide, wherein each of the one or more fluidic cells including a surface having a portion thereof sealed to the coated region, the surface including a depression therein defining a fluidic channel bounded at least in part by the optically exposed region, and a sample introduction port for the introduction of a fluid sample into the fluidic channel; (3) introducing a fluid sample into the fluidic channel via the sample introduction port so that the fluid sample physically contacts the optically exposed region; (4) launching light into the waveguide so as to produce a wave at the optically exposed region; and (5) detecting an optical signal generated at the optically exposed region in response to the wave, wherein the optical signal is correlated with the presence of a prion protein in the fluid sample. An example waveguide device comprises a multimode waveguide having a surface-bearing patterned reflective coating defining a reflectively coated region and an optically exposed region on the surface, wherein one or more first antibodies are covalently bonded to or non-covalently immobilized on the optically exposed region, and wherein the one or more first antibodies selectively binds a prion protein.

### CROSS REFERENCE TO RELATED APPLICATIONS

This application claims the benefit of U.S. Ser. No. 60/774,345 (filed Feb. 17, 2006) and U.S. Ser. No. 60/779,620 (filed Mar. 6, 2006), each of which is incorporated herein by reference in its entirety.

### STATEMENT Regarding FEDERALLY SPONSORED RESEARCH OR DEVELOPMENT

This invention is related to work conducted under United States Navy Contract Nos. NRL-LIC-05-12-178 and NCRADA-NRL-05-366. The government of the United States may have certain rights to the invention.

### FIELD OF THE INVENTION

In an example embodiment, the present invention is directed to a waveguide device for surface-sensitive optical detection of a prion protein in a fluid sample comprising a multimode waveguide having a surface-bearing patterned reflective coating defining a reflectively coated region and an optically exposed region on the surface, wherein one or more first antibodies are covalently bonded to or non-covalently immobilized on the optically exposed region, and wherein the one or more first antibodies selectively binds a prion protein. Another embodiment of the invention includes a method of performing an assay, comprising the steps of (1) providing a multimode waveguide of the invention; (2) fixing one or more fluidic cells to the multimode waveguide, wherein each of the one or more fluidic cells including a surface having a portion thereof sealed to the coated region, the surface including a depression therein defining a fluidic channel bounded at least in part by the optically exposed region, and a sample introduction port for the introduction of a fluid sample into the fluidic channel; (3) introducing a fluid sample into the fluidic channel via the sample introduction port so that the fluid sample physically contacts the optically exposed region; (4) launching light into the waveguide so as to produce a wave at the optically exposed region; and (5) detecting an optical signal generated at the optically exposed region in response to the wave, wherein the optical signal is correlated with the presence of a prion protein in the fluid sample.

### BACKGROUND OF THE INVENTION

Prion diseases or transmissible spongiform encephalopathies (TSEs) are a family of rare progressive neurodegenerative disorders that affect both humans and animals. They are distinguished by long incubation periods, characteristic spongiform changes associated with neuronal loss, and a failure to induce inflammatory response. The causative agent of TSEs is believed to be a prion. A prion is an abnormal, transmissible agent that is able to induce abnormal folding of normal cellular prion proteins in the brain, leading to brain damage and the characteristics signs and symptoms of the disease. Prion diseases are usually rapidly progressive and always fatal. Known human prion diseases include Creutzfeldt-Jakob Disease (CJD), variant Creutzfeldt-Jakob Disease (vCJD), Gerstmann-Straussler-Scheinker Syndrome, fatal familial insomnia, and kuru. Known animal prion diseases include bovine spongiform encephalopathy (BSE, also known as “mad cow” disease), chronic wasting disease (CWD), scrapie, transmissible mink encephalopathy, feline spongiform encephalopathy, and ungulate spongiform encephalopathy.

When a prion disease is discovered in one farm animal, often the entire herd is slaughtered and destroyed to prevent the spread of the disease. In such cases, interest in preventing the spread of prion diseases to humans outweigh the economic losses of the affected farmer. Similarly, when a prion disease is discovered in one country, trade in animal products from that country is often completely embargoed until the health risk abates. Notwithstanding such radical public health measure, the risk of human exposure to prion diseases remains.

According to one known assay for detecting the presence of a prion protein, a sample is treated with a compound that hydrolyzes non-disease related conformation of a protein but partially hydrolyzes or denatures the disease conformation of a protein (i.e., the prion protein), followed by a step of partially denaturing the proteins within the sample. For example, proteinase-K may be used to remove normal protein from a biological sample, so that the sample may be analyzed by immunochromatography to determine the presence and concentration of abnormal (or pathogenic) prion protein. Such methods are time-consuming, require large quantities of chromatography materials and solvents, and they must be carried out by skilled experts.

A need exists for a convenient, rapid, and inexpensive method of testing samples, such as biological materials, for the presence of prion proteins.

### SUMMARY OF THE INVENTION

The present invention provides materials, devices, and methods that overcome the above-noted deficiencies of the prior art. For example, an embodiment of the invention includes a method of performing an assay of a biological material comprising the steps of (1) providing a multimode waveguide having a surface-bearing patterned reflective coating defining a reflectively coated region and an optically exposed region on said surface, the optically exposed region generating an optical signal indicative of the presence of a prion protein in a fluid sample in response to a wave at the surface, wherein the optically exposed region is bonded with an antibody that selectively binds a prion protein; (2) fixing one or more fluidic cells to the multimode waveguide, wherein each of the one or more fluidic cells including a surface having a portion thereof sealed to the coated region, the surface including a depression therein defining a fluidic channel bounded at least in part by the optically exposed region, and a sample introduction port for the introduction of a fluid sample into the fluidic channel; (3) introducing a fluid sample into the fluidic channel via the sample introduction port so that the fluid sample physically contacts the optically exposed region; (4) launching light into the waveguide so as to produce a wave at the optically exposed region; and (5) detecting an optical signal generated at the optically exposed region in response to the wave, wherein the optical signal is correlated with the presence of a prion protein in the fluid sample.

The method may further comprise a step of introducing a tracer solution comprising a prion indicator, e.g., one or more antibodies that selectively bind a prion protein, wherein the antibody produces an alteration of the optically exposed region, the alteration being detectable by launching light into the waveguide to generate a wave at the surface, and then detecting an interaction of the optically exposed region with the wave. The prion indicator may contain one or more antibodies that are covalently bonded to a fluorophore or dye, such as fluorescein, rhodamine, hydroxycoumarin, digoxigenin, cyanine, diazaindacene, or a combination or derivative thereof, and other compounds that function in a similar manner.

An example waveguide device of the invention comprises a multimode waveguide having a surface-bearing patterned reflective coating defining a reflectively coated region and an optically exposed region on the surface, wherein one or more antibodies are covalently bonded to or non-covalently immobilized on the optically exposed region, and wherein the one or more antibodies selectively binds a prion protein.

Example biological materials that may be analyzed according to an embodiment of the invention include eyelid, blood, plasma, cerebrospinal fluid, neurological tissue, lymph, saliva, semen, feces, urine, aqueous humor, muscle, offal, or a combination, mixture, homogenate, extract, concentrate, or component thereof. Example prion proteins include the pathogenic proteins associated with chronic wasting disease (CWD), bovine spongiform encephalopathy (BSE), kuru, Creutzfeldt-Jakob disease (CJD), variant Creutzfeldt-Jakob Disease (vCJD), Gerstmann-Straussler-Scheinker Syndrome, fatal familial insomnia, scrapie, transmissible mink encephalopathy, feline spongiform encephalopathy, and ungulate spongiform encephalopathy.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a not to scale, cross-sectional view of a reflectively coated multimode waveguide device having a fluidics cell attached thereto, according to an embodiment of the invention.

FIG. 2 is a not to scale, cross-sectional side view of an example waveguide device of an embodiment of the invention, coupled with a light source.

FIG. 3 is a not to scale cross-sectional top view of a waveguide device according to an embodiment of the invention.

FIG. 4 is not to scale and illustrates an example multi-channel fluidics system according to an embodiment of the invention.

FIG. 5 is not to scale and shows an example optical system for use with an embodiment of the invention.

FIG. 6 is not to scale and shows how excitation may be achieved by using a lens to focus light into a waveguide and allowing the beam to propagate.

FIG. 7 is an example waveguide device according to an embodiment of the invention, including a fluid pumping system.

FIG. 8 illustrates an embodiment of the invention where different antibodies are used to selectively bind different prions or recognition elements.

### DETAILED DESCRIPTION OF THE INVENTION

Accordingly, an embodiment of the invention provides a waveguide device for surface-sensitive optical detection of a prion protein in a fluid sample comprising a multimode waveguide having a surface-bearing patterned reflective coating defining a reflectively coated region and an optically exposed region on the surface, wherein one or more first antibodies are covalently bonded to or non-covalently immobilized on the optically exposed region, and wherein the one or more first antibodies selectively binds a prion protein. The optically exposed region is sensitive to a prion protein so as to produce an alteration of the optically exposed region indicative of the presence of the prion protein in a fluid sample, the alteration being detectable by launching light into the waveguide to generate a wave at the surface, and then detecting an interaction of the optically exposed region with the wave. The wave may be an evanescent wave or an electromagnetic wave that has transitioned into a transmitted regime as scattered light or other modes. In an example embodiment, the wave is detectable within a distance from the surface of the waveguide and encompassing all immobilized materials. For example, the wave may be detectable at a distance of about 50 nm from the surface of the waveguide, or at about 500 nm, or 100 nm, or even several microns from the optically exposed region of the surface.

According to another embodiment of the invention, the waveguide may have any shape, for example cylindrical (e.g., a rod) or planar. Typically, the waveguide used in the invention is planar. The waveguide may be made of any material that transmits light at both the excitation wavelength and the signal wavelength or wavelengths. For example, the waveguide may be an inorganic glass or a solid such as a polymer (e.g., a plastic such as polystyrene).

In yet another embodiment of the invention, the reflective coating is comprised of gold, silver, aluminum, platinum, rhodium, a dielectric, chromium, any other metal or a mixture thereof, although the reflective coating may be any material that reflects light at the excitation wavelength. Additionally, the reflective coating may be a multilayer dichroic mirror. The bonding of the reflective coating to the waveguide may be enhanced by providing the reflective coating as a multilayered structure including a reflective layer, which may be the aforementioned reflective metal or dichroic mirror, and a bonding layer between the reflective layer and the waveguide. This bonding layer is selected to enhance adhesion as compared with direct bonding between the waveguide surface and the reflective layer. Preferably, the bonding layer is selected to have minimal or no scattering or absorption of the excitation light. Typical materials for the bonding layer include chromium, platinum, rhodium, a dielectric, a silane (particularly a thiol silane), a cyanoacrylate, a polymer, or a mixture thereof. If desired, the outer surface of the reflective layer (used by itself or as part of multilayer structure with the bonding layer) may be provided with a protective coating to protect the reflective layer from chemical or mechanical damage. Typical materials for the protective coating include chromium, platinum, rhodium, a dielectric, a polymer, or a mixture thereof. A reflective coating may be applied to the surface of the waveguide according to a variety of art-recognized techniques. Typical methods for patterning metal or other reflective coatings on glass or plastic substrates include masked vacuum evaporation of the reflective coating, photolithography, and vapor deposition, among others. The same or similar processes may be used to provide the reflective coating as a multilayer structure.

The optically exposed regions of the patterned waveguide surface are sensitive, i.e., responsive, to at least one prion protein so that direct or indirect interaction of the optically exposed regions with at least one prion-containing analyte alters an optically exposed region. This alteration may be directly or indirectly detectable by launching a wave, e.g., light, into the waveguide. In an embodiment of the invention, the surface of an optically exposed region of a waveguide is made sensitive to at least one prion protein by being coated with an anti-prion antibody layer that specifically binds a prion protein. Typical methods for attaching antibody recognition species to surfaces include covalent binding, physisorption, biotin-avidin binding, or modification of the surface with a thiol-terminated silane/heterobifunctional crosslinker, among others.

If the reflective coating is applied before attachment of an anti-prion antibody, particular care should be taken to assure that the antibodies are immobilized to the optically exposed waveguide regions of the waveguide surface under conditions that maintain the integrity of the reflectively coated portions. Any protocol for the attachment of antibodies to the surface of the waveguide should ideally avoid delamination or other destructive modification of the reflective coating. In the biotin-avidin and thiol silane methods, avoiding delamination and destructive modification of the reflective coating typically requires that all solutions to which the reflective coating is exposed during attachment of the antibodies to have a salt concentration significantly below the physiological salt concentration (typically about 150 mM). If the salt concentration is too high, delamination may result. If the salt concentration is too low, the antibodies may lose their functionality. Furthermore, at any given salt concentration, the extent of delamination may be reduced by performing the attachment chemistries at lower temperature (above freezing, of course). However, low temperatures during immobilization may increase the time required for the binding of the antibodies to the waveguide surface. Typically, immobilization of the antibodies to the waveguide surface is performed from between ambient temperatures and about 4° C. Determining optimal antibody immobilization methodology is within the scope of routine experimentation that may be carried out by one of skill in the art in accordance with the principles described herein.

When immobilizing anti-prion antibodies to a patterned waveguide, antibodies may also be attached to the reflective coating. However, because the reflective coating will be covered with the fluidics cell and be optically inactive, this attachment is not generally of significant concern. In some instances, such as where an antibody is particularly expensive, it may be advantageous to use other molecular patterning technologies such as a contact patterning or a non-contact patterning method, e.g., stamping or inkjet printing to attach the antibody molecules only on the optically exposed regions of the waveguide surface.

According to one embodiment of the invention, the immobilized antibody, which may be monoclonal or polyclonal, selectively binds a polypeptide comprising a sequence of amino acids selected from the group consisting of Gly-Gln-Gly-Gly-Gly-Thr-His-Ser-Gln-Trp-Asn-Lys-Pro-Ser (SEQ ID NO: 1), Gly-Gln-Gly-Gly-Ser-His-Ser-Gln-Trp-Asn-Lys-Pro-Ser (SEQ ID NO: 2), Ser-Asp-Tyr-Glu-Asp-Arg-Tyr-Tyr-Arg-Glu-Asn-Met-His-Arg (SEQ ID NO: 3), Asn-Asp-Tyr-Glu-Asp-Arg-Tyr-Tyr-Arg-Glu-Asn-Met-Tyr-Arg (SEQ ID NO: 4), Lys-Thr-Asn-Met-Lys-His-Val-Ala-Gly-Ala-Ala-Ala-Ala-Gly-Ala-Val-Val-Gly-Gly-Leu-Gly (SEQ ID NO: 5), Arg-Tyr-Pro-Asn-Gln-Val-Tyr-Tyr-Arg-Pro-Val-Asp-Arg-Tyr-Ser-Asn-Gln-Asn-Asn-Phe-Val-His-Asp (SEQ ID NO: 6), Arg-Glu-Ser-Gln-Ala-Tyr-Tyr-Gln-Arg-Gly-Ser-Ser-Met-Val-Leu (SEQ ID NO: 7), and Arg-Glu-Ser-Gln-Ala-Tyr-Tyr-Gln-Arg-Gly-Ala-Ser-Val-Ile-Leu (SEQ ID NO: 8). Similarly, a plurality of different antibodies may be immobilized on the waveguide in particular addressable locations on the surface of the waveguide.

In another embodiment, the invention includes a waveguide coupled to a fluidic cell including a surface having a portion thereof sealed to the coated region, the surface including a depression therein defining one or more fluidic channels bounded at least in part by the optically exposed region; and a sample introduction port for the introduction of a fluid sample into each of the one or more fluidic channels. The fluidic cell may be made of any material compatible with the fluids employed during operation. Typically, the fluidic cell is made of a polymer such as polymethylmethacrylate, polycarbonate, or polystyrene. The fluidic cell should be capable of forming a fluid-tight seal with the reflectively coated portion of the waveguide, either with or without the assistance of an adhesive or a gasket. The fluidic cell may be either rigid or elastic, and may be a single material or a composite or multilayer structure. In the case of a fluidic cell that is adhered to the waveguide by pressure, without the use of an adhesive, it may be advantageous for the surface of the fluidic cell in contact with the reflectively coated portion of the waveguide to be elastic so as to facilitate the formation of a fluid-tight seal. If the fluidic cell is attached to the reflective coating of the waveguide with the assistance of an adhesive, the adhesive should be compatible with the fluidic cell, the reflective coating, and the fluids employed.

In yet another embodiment, the present invention allows the attachment of the waveguide to other components, such as optical elements (including light sources, detectors, lenses, filters, etc.), or mechanical elements (such as mounts, pumps, valves, etc), and electronic elements (such as transistors, microcircuits, displays, etc.) used in optically-transduced assays without significantly optically perturbing the light-guiding characteristics of the waveguide. By attaching such components or one or more mounts for such components, for example, attaching the mounts or other components by the use of an adhesive, to the reflectively clad region or regions of the waveguide surface, the optical characteristics of the waveguide will be essentially unperturbed while gaining the additional functionality of the attached component. These additional components may be attached to the waveguide in addition to or instead of a fluidic cell.

Accordingly, an embodiment of the invention includes a light source optically coupled into the waveguide so as to produce a wave at the optically exposed region. The light source may be one or more lasers, each having a wavelength of from about 100 nm to about 3000 nm. Similarly, the light source may be polychromatic. The particular light source should be selected so that the perturbation in the resulting wave within the waveguide device is detectable when in operation. Therefore, another embodiment of the invention includes a detector that detects an optical signal generated at the optically exposed region in response to the light source, such as a CCD camera, a CCD chip, or an electronically amplified CCD chip, among others.

In another embodiment, the invention provides a method of performing an assay, comprising the steps of (1) providing a multimode waveguide having a surface-bearing patterned reflective coating defining a reflectively coated region and an optically exposed region on the surface, the optically exposed region generating an optical signal indicative of the presence of a prion protein in a fluid sample in response to a wave at the surface, wherein the optically exposed region is bonded with a first antibody that selectively binds a prion protein; (2) fixing one or more fluidic cells to the multimode waveguide, wherein each of the one or more fluidic cells including a surface having a portion thereof sealed to the coated region, the surface including a depression therein defining a fluidic channel bounded at least in part by the optically exposed region, and a sample introduction port for the introduction of a fluid sample into the fluidic channel; (3) introducing a fluid sample into the fluidic channel via the sample introduction port so that the fluid sample physically contacts the optically exposed region; (4) launching light into the waveguide so as to produce a wave at the optically exposed region; and (5) detecting an optical signal generated at the optically exposed region in response to the wave, wherein the optical signal is correlated with the presence of a prion protein in the fluid sample. Prior to or subsequent to any of these enumerated steps, the method may optionally include an additional step of introducing a buffer solution into the fluidic channel via the sample introduction port to remove interfering material from the fluidic cell. Interfering materials may comprise a non-prion protein, cellular debris, or a non-protein materials, among others.

In an example embodiment, a buffer solution may comprise water and a water-soluble salt-based buffer, such as phosphate buffered saline. A buffer may also comprise a detergent, such as a polysorbate detergent, e.g., TWEEN® (a registered trademark of ICI Americas Inc. of Bridgewater, N.J.). A buffer solution may also comprise a blocking agent that binds to non-specific locations within the fluidic channel or any fluid conduit or pump connected thereto. An example blocking agent is bovine serum albumin (BSA), which is known to be “sticky” and is used according to the invention to reduce or eliminate non-specific interactions, such as protein-protein interactions or protein-surface interactions.

In an embodiment of the invention, a tracer solution is introduced into the fluidic channel via the sample introduction port, wherein the tracer solution comprises a prion indicator. A prion indicator is any material that causes a detectable perturbation indicative of the presence of a prion protein at an optically exposed region of the waveguide during operation. The prion indicator is selectively retained by the immobilized antibodies. The prion indicator may be one or more peptides, e.g., peptides according to SEQ ID NOS: 1-8, optionally conjugated to a fluorophore or dye. After the tracer solution has been introduced, a fluid sample is introduced. If the prion indicator is a peptide conjugated to a fluorophore, then a prion protein in the sample is detectable by launching light into the waveguide to generate a wave at the surface, and then detecting a reduction of the optical signal at the optically exposed region. According to another embodiment of the invention, after introducing a fluid sample to a waveguide device, a tracer solution may be introduced into the fluidic channel via the sample introduction port, wherein the tracer solution comprises a prion indicator. The prion indicator may be one or more antibodies that selectively bind a prion protein, wherein the second antibody produces an alteration of the optically exposed region, the alteration being detectable by launching light into the waveguide to generate a wave at the surface, and then detecting an interaction of the optically exposed region with the wave. The prion indicator (e.g., antibody or peptide) may be covalently bonded to a fluorophore or dye, such as fluorescein, rhodamine, hydroxycoumarin, digoxigenin, cyanine, diazaindacene, and other compounds that function in a similar manner, or combinations and derivatives thereof. An example class of diazaindacene fluorophores are BODIPY® fluorophores, and some example cyanine-like fluorophores are among the ALEXAFLUOR® fluorophores, which are commercially available from Invitrogen, Corp. (Carlsbad, Calif.). (BODIPY® and ALEXAFLUOR® are both registered trademarks of Molecular Probes, Inc. of Eugene, Oreg.).

The one or more first antibodies that are described herein above and are immobilized on the waveguide may be different from the one or more second antibodies that are used as a prion indicator. The prion indicator may be a second antibody that selectively binds a polypeptide comprising a sequence of amino acids selected from the group consisting of Gly-Gln-Gly-Gly-Gly-Thr-His-Ser-Gln-Trp-Asn-Lys-Pro-Ser (SEQ ID NO: 1), Gly-Gln-Gly-Gly-Ser-His-Ser-Gln-Trp-Asn-Lys-Pro-Ser (SEQ ID NO: 2), Ser-Asp-Tyr-Glu-Asp-Arg-Tyr-Tyr-Arg-Glu-Asn-Met-His-Arg (SEQ ID NO: 3), Asn-Asp-Tyr-Glu-Asp-Arg-Tyr-Tyr-Arg-Glu-Asn-Met-Tyr-Arg (SEQ ID NO: 4), Lys-Thr-Asn-Met-Lys-His-Val-Ala-Gly-Ala-Ala-Ala-Ala-Gly-Ala-Val-Val-Gly-Gly-Leu-Gly (SEQ ID NO: 5), Arg-Tyr-Pro-Asn-Gln-Val-Tyr-Tyr-Arg-Pro-Val-Asp-Arg-Tyr-Ser-Asn-Gln-Asn-Asn-Phe-Val-His-Asp (SEQ ID NO: 6), Arg-Glu-Ser-Gln-Ala-Tyr-Tyr-Gln-Arg-Gly-Ser-Ser-Met-Val-Leu (SEQ ID NO: 7), and Arg-Glu-Ser-Gln-Ala-Tyr-Tyr-Gln-Arg-Gly-Ala-Ser-Val-Ile-Leu (SEQ ID NO: 8).

Furthermore, the one or more first (immobilized) antibodies and the one or more second (prion indicator) antibodies may each be polyclonal antibodies, monoclonal antibodies, or a combination thereof. In either case, the antibodies may be derived from animal antisera (e.g., rabbit, goat, sheep, bovine, or primate/human, among others), and in an advantageous embodiment the antibodies bind a prion protein associated with a prion disease, such as chronic wasting disease (CWD), bovine spongiform encephalopathy (BSE), kuru, Creutzfeldt-Jakob disease (CJD), variant Creutzfeldt-Jakob Disease (vCJD), Gerstmann-Straussler-Scheinker Syndrome, fatal familial insomnia, scrapie, transmissible mink encephalopathy, feline spongiform encephalopathy, or ungulate spongiform encephalopathy.

According to another embodiment of the invention, the fluid sample that is to be analyzed according by any device of the invention or any method relating thereto may be biological material, such as eyelid, blood, plasma, cerebrospinal fluid, neurological tissue, lymph, saliva, semen, feces, urine, aqueous humor, muscle, offal, or a combination, mixture, homogenate, extract, concentrate, or component thereof. In addition, the fluid sample may also comprise water or an aqueous buffer or a carrier.

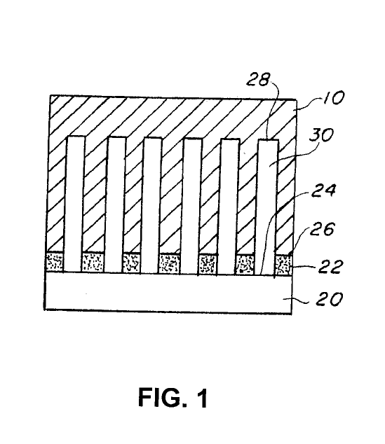

Referring to the accompanying Drawings, FIG. 1 depicts an embodiment of the invention illustrated by a cross-sectional view of an example fluidics cell 10 attached to a waveguide 20 having a patterned reflective coating thereon. Waveguide 20 includes a patterned reflective coating 22 on its surface that leaves optically exposed regions 24. Bottom surface 26 of fluidics cell 10 has depressions 28 formed therein. These depressions form fluidic channels 30 that are bounded in part by optically exposed regions 24 on the upper surface of waveguide 20. Each fluidic channel 30 has a sample introduction port 65 (shown in FIG. 3). Because each fluidic channel 30 is independent (between fluidic channels 30, bottom surface 26 of fluidics cell 10 forms a seal with reflective coating 22 or an adhesive between them) multiple samples may be analyzed simultaneously.

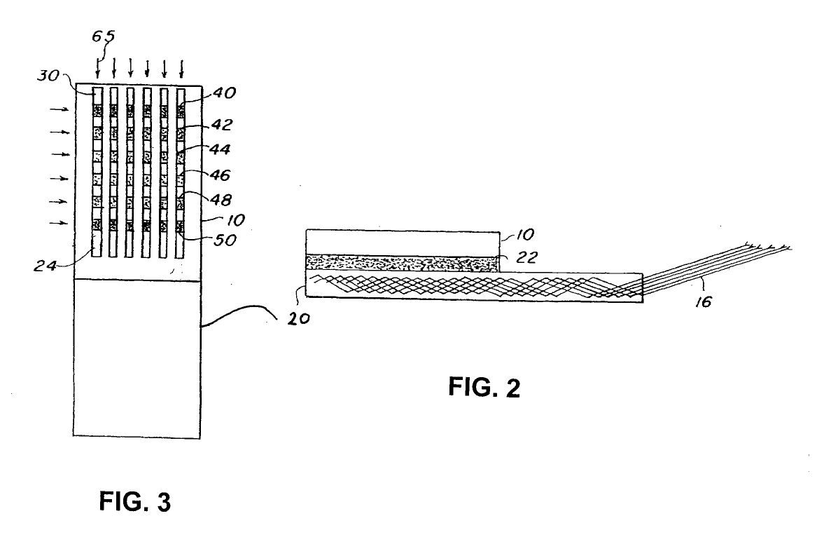

FIG. 2 is a side view of a waveguide device according to an embodiment of the invention, and illustrates the propagation of light 16 into a waveguide 20 after attachment of a fluidics cell 10 because the reflective coating 22, patterned to match or extend beyond the contact points of fluidics cell 10, eliminates the out-coupling of light into the fluidics cell 10. In this manner, it is possible to attach fluidics cell 10 to the waveguide and perform optical measurements before, during, and after exposure to samples introduced through flow channels in fluidics cell 10.

FIG. 3 is a top view of a cross-section of an assembled waveguide device according to an embodiment of the invention. Six stripes 40, 42, 44, 46, 48, and 50 extend along the width of the surface of waveguide 20, across both the reflectively coated regions and the optically exposed regions. The six stripes may comprise anti-prion antibodies specific for the same prion protein, or may comprise anti-prion antibodies for different prion proteins. Accordingly, an embodiment of the invention provides for simultaneous assaying for different analytes. Attached fluidics cell 10 covers the patterned reflective coating (not visible through fluidics cell 10). Together exposed regions 24 of waveguide 20 and fluidics cell 10 form flow channels 30. Each flow channel 30 may include a separate sample introduction port 65. Thus, using the device illustrated in the FIG. 3, six different samples may be simultaneously assayed for the presence of six different prion proteins or recognition elements (e.g., variable regions). Of course, one skilled in the art will readily appreciate that different configurations of the invention are possible, including waveguide devices with one, two, three, four, five, seven, eight, or even as many as 16 to 33 stripes of anti-prion antibodies, and one, two, three, four, five, seven, eight, or even as many as 16 to 33 fluid flow channels. If microfabrication techniques are employed, up to 100 stripes of anti-prion antibodies or fluid flow channels may be obtained.

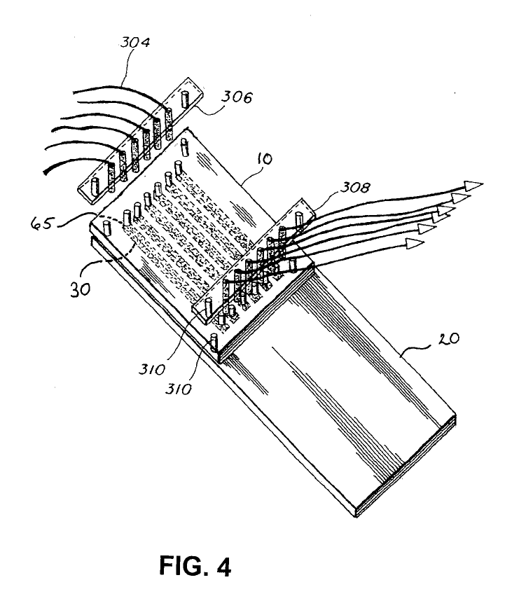

According to an exemplary embodiment as depicted in FIG. 4, fluidic connections between an automated dispensing system (not depicted) and a flow cell 10 is accomplished using an inlet manifold 306 comprising a multiplicity of fluid fittings 304 and a gasket (not depicted) to make fluid tight seals to the inlet manifold 306 ports and outlet manifold 308 ports with the fluidics cell 10. In order to replace such a six-channel analysis unit, the four complementary pairs of mated connecting means 310 should be adjusted.

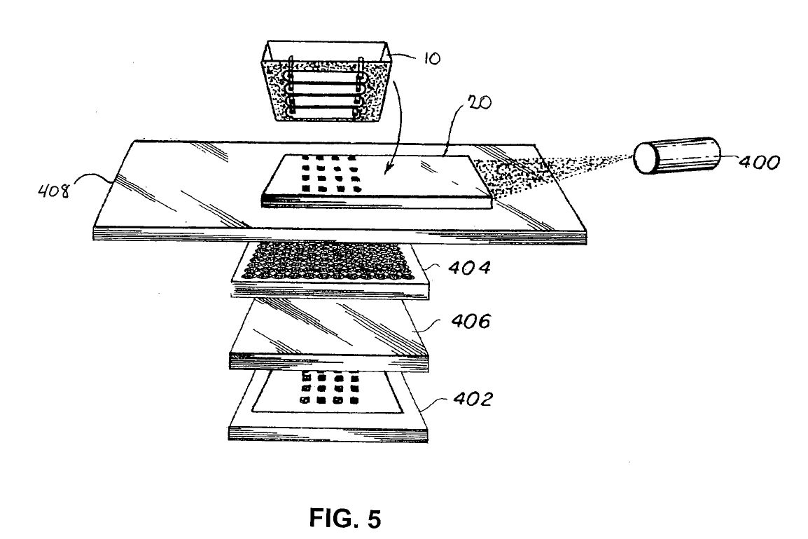

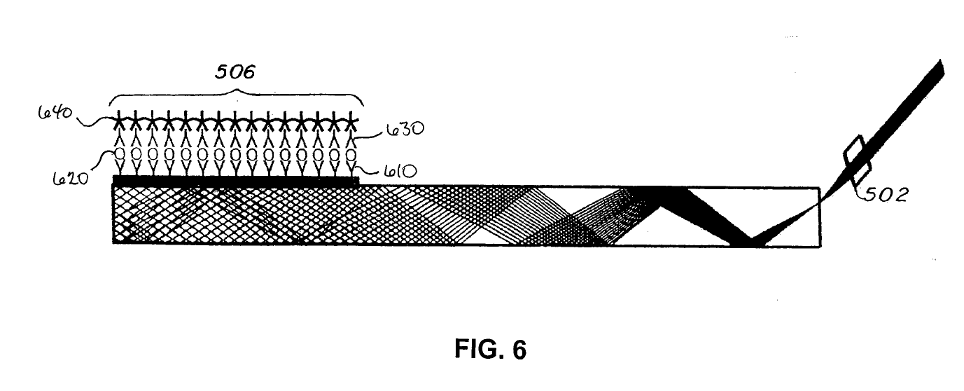

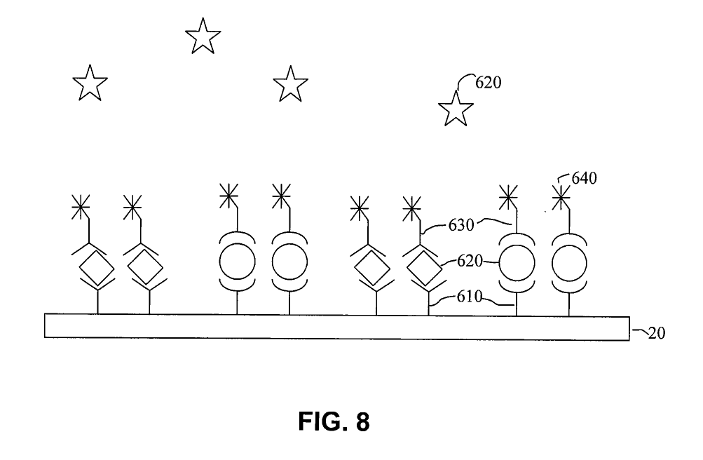

In the embodiment of the invention shown in FIG. 5, a beam of light, e.g., from a diode laser 400 is launched into the edge of a waveguide 20 (e.g., a standard microscope slide) that is mounted on a mounting bracket 408, evenly illuminating the entire lateral width of the waveguide 20. To examine the fluorescent pattern from the detection assays, a compact imaging system may be used to record the spatial orientation of the fluorescent array elements. For example, an excited fluorescent pattern may be imaged onto a thermoelectrically cooled charge-coupled device (CCD) imaging array 402, optionally in conjunction with a filter 406, lens array 404, or similar image modifying objects. An optional cylindrical lens 502 (FIG. 6) may also be used to provide uniform longitudinal excitation at the sensing region 506, although a mirror 700 (FIG. 6) may also be used to focus the light 16. As shown in FIG. 6, a light beam diverges after it is focused onto the proximal end of a guide and spreads out within the waveguide prior to the sensing region 506. Immobilized on the surface of the sensing region are anti-prion antibodies 610 coupled with prion protein 620 and a second anti-prion antibody 630 having a fluorescent label 640. The anti-prion antibodies 610, 630 selectively recognize and interact with prion protein(s) 620 as further illustrated in FIG. 8.

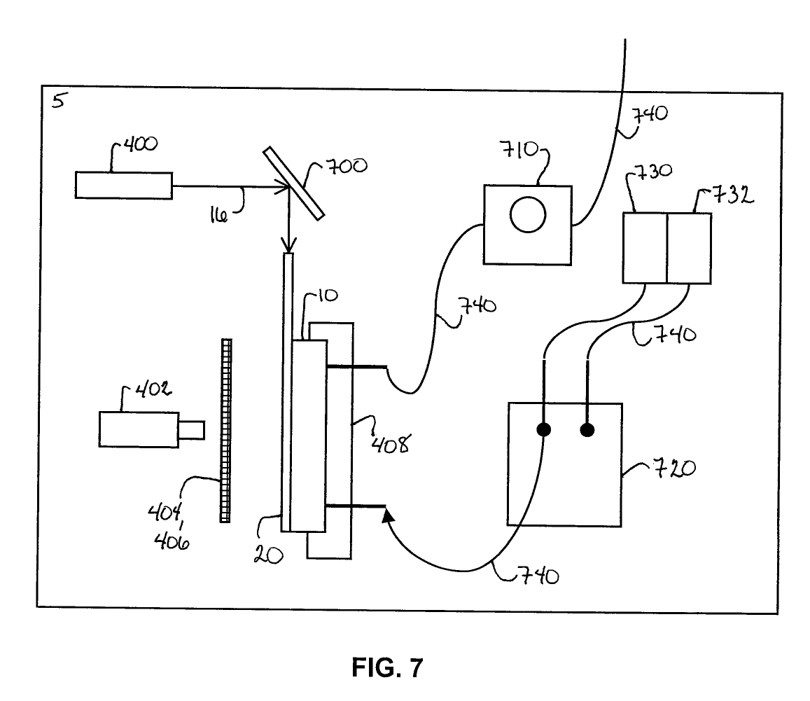

FIG. 7 illustrates an example waveguide device 5 according to an embodiment of the invention. A fluid sample to be analyzed is contained within a sample reservoir 730 and a solution containing a tracer solution is contained within a prion indicator reservoir 732. The two reservoirs are in fluid communication with a valve switching means 720, a fluidics cell 10, and a pumping means 710 by, e.g., a non-reactive tubing material 740. During operation of the device, a portion of the sample and then a portion of the prion indicator solutions are pumped through the fluidics cell 10, while light 16 produced by a laser 400 is focused into a waveguide 10 by means of a mirror 700. The presence of a prion protein in the sample, e.g., by fluorescence, is detected by a CCD camera 402, optionally with one or more lenses 404 or filters 406. As illustrated in FIG. 7, the tubing material 740 may be connected to a solvent reservoir or fluid waste receptacle (not illustrated) that is external to the waveguide device 5.

FIG. 8 illustrates an embodiment of the invention where different antibodies are used to selectively bind different prions or recognition elements. Two or more different first antibodies 610, each selective for a different peptide sequence, are covalently and/or non-covalently attached to the waveguide 20. Each antibody 610 selectively recognizes its specific target sequence or prion 620. Two or more different second antibodies 630, each selective for a different peptide sequence or prion and labeled with a fluorophore 640, are shown as a sandwich with their specific targets 620. Only peptide sequences or prions 620 that are selectively recognized by the antibodies 610, 630 will be detected.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. The invention is further illustrated by the foregoing examples, which should not be construed as further limiting.

### EXAMPLES

Two rabbit polyclonal antibodies were made by conjugation of the following two peptides to a carrier protein: Ser-Asp-Tyr-Glu-Asp-Arg-Tyr-Tyr-Arg-Glu-Asn-Met-His-Arg (SEQ ID NO: 3) and Arg-Glu-Ser-Gln-Ala-Tyr-Tyr-Gln-Arg-Gly-Ala-Ser-Val-Ile-Leu (SEQ ID NO: 8). Three rabbits were immunized on day 0. At day 14, day 42, and day 56 the rabbits were re-immunized, and at day 52, day 66, and day 70 the rabbits were bled to produce approximately 150 mL of crude serum. Affinity chromatography with a stationary phase containing the antigenic peptide produced approximately 5 mL of crude serum, which was assayed by SDS-PAGE.

Biotinylated first antibodies were prepared by combining antibody (0.5 mg/50 μL phosphate buffered saline) in bicarbonate buffer, pH 8 (450 μL) with biotin (75.76 μL of 1 mg/ml in dimethylsulfoxide) for 30 minutes at room temperature. Biotinylated first antibodies were isolated from unconjugated biotin using a MW10000 cutoff size exclusion column.

Prion indicator second antibodies were prepared by combining antibody (0.5 mg/50 μL phosphate buffered saline) with dye (AlexaFluor 647®, 50 μg+5 μL dimethylsulfoxide+5 μL water) for one hour at room temperature in the dark. Tracer conjugated second antibodies were isolated from unconjugated dye using a MW10000 cutoff size exclusion column.

Patterned waveguides were made in two phases. The first phase placed a uniform adherence layer on the waveguide. The second phase placed the first antibody in discrete locations for the assay. Phase 1 was a multistep process where waveguides were (1) cleaned and prepared for modification by immersion in a potassium hydroxide and methanol bath for 30 minutes at room temperature, (2) functionalized by reacting with a silane (8 g of 3-mercaptopropyl triethoxysilane in 80 mL of toluene) for 1 hour at room temperature under a nitrogen atmosphere, (3) crosslinked by incubating with succinimidyl 4-maleimidobutyrate (GMBS, 12 mg in 250 μL dimethylsulfoxide and 45 mL ethanol) for 30 minutes at room temperature, and (4) functionalized by reacting with NeutrAvidin® (3 mg in 30 mL of phosphate buffer) for 2 hours at room temperature. (NEUTRAVIDIN®, a deglycosylated form of avidin, is a registered trademark of Pierce Biotechnology, Inc. of Rockford, Ill.). Phase 2 was also a multistep process where (1) the patterning gasket was treated with 10% bovine serum albumin in phosphate buffered saline with Tween® to eliminate or reduce non-specific binding to the gasket, (2) the waveguide functionalized in Phase 1 and the blocked patterning gasket were layered into and immobilized in a patterning assembly, (3) first antibody solutions were introduced by syringe into fluidics channels formed by the patterning gasket pressed against the functionalized waveguide and allowed to sit in contact for a minimum of 4 hours at 4° C., (4) the fluidics channels were cleared of the first antibody solutions and rinsed with a blocking phosphate buffered solution with Tween® and bovine serum albumin, (5) the patterning assembly was disassembled and the patterned waveguide was immersed in a blocking phosphate buffered solution with Tween® and bovine serum albumin for 10 minutes at room temperature then rinsed with 18.5 MΩ water and dried under a nitrogen stream.

To perform the assay, the patterned waveguide was placed in the waveguide device and locked in contact with the fluidics cell. In the following sequence, fluids were run through the device to bring each fluid in contact with the length of the patterned waveguide: (1) 800 μL of phosphate buffered saline with Tween 20® and bovine serum albumin through both sample and prion indicator reservoirs, (2) 800 μL of recombinant prion sample (10 ug/ml) through the sample reservoir, (3) 800 μL buffer through the sample reservoir, (4) 400 μL of prion indicator second antibody (10 ug/ml) through the prion indicator reservoir, and (5) 800 μL of buffer through the prion indicator reservoir. The buffer was used to remove interfering materials and to reduce or remove non-specific binding of the sample. To generate the signal used in detection, light from a 635 nm laser was launched into the proximal end of the waveguide. As the light traveled the length of the waveguide, it formed an evanescent wave providing energy to the first 500 nm into the sensing surface as well as transitioning into a transmitted regime of energy as scattered light or other modes. The energy provided from these light sources energized the prion indicator antibody which released or emitted a portion of the energy as fluorescent light. The fluorescent light was detected in the infrared range and was subsequently detected by the camera equipped with the appropriate filter set in the device. Images were then collected and analyzed using the device to demonstrate recombinant prion detection. According to the patterned grid, recombinant prion was detected in the appropriate locations as indicated by the captured emitted light. This demonstrated that the antibody combinations detected the recombinant prion protein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A waveguide device for surface-sensitive optical detection of a prion protein in a fluid sample comprising a multimode waveguide having a surface-bearing patterned reflective coating defining a reflectively coated region and an optically exposed region on said surface, wherein one or more first antibodies are covalently bonded to or non-covalently immobilized on said optically exposed region, and wherein said one or more first antibodies selectively binds a prion protein.

2. The waveguide device of claim 1, wherein said optically exposed region is sensitive to a prion protein so as to produce an alteration of said optically exposed region indicative of the presence of said prion protein in a fluid sample, said alteration being detectable by launching light into said waveguide to generate a wave at said surface, and then detecting an interaction of said optically exposed region with said wave.

3. The waveguide device of claim 2, wherein said wave is an evanescent wave or an electromagnetic wave that has transitioned into a transmitted regime as scattered light or other modes.

4. The waveguide device of claim 3, wherein said wave is detectable at a distance from the surface of the waveguide that encompassed all immobilized materials.

5. The waveguide device of claim 3, wherein said wave is detectable within 500 nm of said optically exposed region of said surface.

6. The waveguide device of claim 1, wherein said waveguide is comprised of glass or a solid polymer.

7. The waveguide device of claim 1, wherein said reflective coating is comprised of gold, silver, aluminum, platinum, rhodium, a dielectric, chromium, any other metal, or a mixture thereof.

8. The waveguide device of claim 1, further comprising a light source optically coupled into said waveguide so as to produce a wave at said optically exposed region.

9. The waveguide device of claim 8, wherein said light source is one or more lasers or a polychromatic light source.

10. The waveguide device of claim 9, wherein the wavelength of each of said one or more lasers is from about 100 nm to about 3000 nm.

11. The waveguide device of claim 9, wherein said polychromatic light source is a carbon arc lamp or an incandescent light bulb.

12. The waveguide device of claim 8, further comprising a detector that detects an optical signal generated at said optically exposed region in response to said light source.

13. The waveguide device of claim 9, wherein said detector is a CCD camera, a CCD chip, or an electronically amplified CCD chip.

14. The waveguide device of claim 1, further comprising a fluidics cell including a surface having a portion thereof sealed to said coated region, said surface including a depression therein defining one or more fluidic channel bounded at least in part by said optically exposed region; and a sample introduction port for the introduction of a fluid sample into each of said one or more fluidic channels.

15. A method of performing an assay, comprising the steps of:
providing a multimode waveguide having a surface-bearing patterned reflective coating defining a reflectively coated region and an optically exposed region on said surface, said optically exposed region generating an optical signal indicative of the presence of a prion protein in a fluid sample in response to a wave at said surface, wherein said optically exposed region is bonded with a first antibody that selectively binds a prion protein;
fixing one or more fluidic cells to said multimode waveguide, each of said one or more fluidic cells including:
a surface having a portion thereof sealed to said coated region, said surface including a depression therein defining a fluidic channel bounded at least in part by said optically exposed region, and
a sample introduction port for the introduction of a fluid sample into said fluidic channel;
introducing a fluid sample into said fluidic channel via said sample introduction port so that said fluid sample physically contacts said optically exposed region;
launching light into said waveguide so as to produce a wave at said optically exposed region; and
detecting an optical signal generated at said optically exposed region in response to said wave, wherein said optical signal is correlated with the presence of a prion protein in said fluid sample.

16. The method of claim 15 further comprising, before the step of introducing a fluid sample, a step of introducing a first buffer solution into said fluidic channel via said sample introduction port to remove interfering material from said fluidics cell.

17. The method of claim 16, wherein said interfering material comprises a non-prion protein, cellular debris, or a non-protein material.

18. The method of claim 16, wherein said first buffer solution comprises water and a water-soluble salt-based buffer.

19. The method of claim 18, wherein said first buffer solution is phosphate buffered saline.

20. The method of claim 18, wherein said first buffer solution comprises a detergent.

21. The method of claim 20, wherein said detergent is a polysorbate detergent.

22. The method of claim 18, wherein said first buffer solution further comprises a blocking agent that binds to non-specific locations within said fluidic channel or any fluid conduit or pump connected thereto.

23. The method of claim 22, wherein said blocking agent is bovine serum albumin.

24. The method of claim 15 further comprising, after the step of introducing a fluid sample, a subsequent step of introducing a second buffer solution into said fluidic channel via said sample introduction port to remove interfering material from said fluidics cell.

25. The method of claim 15 further comprising, after the step of introducing a fluid sample, a subsequent step of introducing a tracer solution into said fluidic channel via said sample introduction port, wherein said tracer solution comprises a prion indicator.

26. The method of claim 25 further comprising, after the step of introducing a tracer solution, a subsequent step of introducing a third buffer solution into said fluidic channel via said sample introduction port to remove excess prion indicator.

27. The method of claim 18, wherein said prion indicator is one or more second antibodies that selectively bind a prion protein, wherein said second antibody produces an alteration of said optically exposed region, said alteration being detectable by launching light into said waveguide to generate a wave at said surface, and then detecting an interaction of said optically exposed region with said wave.

28. The method of claim 25, wherein said one or more second antibodies are covalently bonded to a fluorophore or dye.

29. The method of claim 28, wherein said fluorophore or dye is fluorescein, rhodamine, hydroxycoumarin, digoxigenin, cyanine, diazaindacene, or a combination or derivative thereof.

30. The method of claim 27, wherein said one or more first antibodies is different from said one or more second antibodies.

31. The method of claim 30, wherein said one or more first antibodies and said one or more second antibodies are polyclonal antibodies, monoclonal antibodies, or a combination thereof.

32. The method of claim 30, wherein said one or more first antibodies or said one or more second antibodies are derived from an animal source.

33. The method of claim 32, wherein said animal is selected from the group consisting of rabbits, goats, sheep, bovines, and primates.

34. The method of claim 31, wherein said one or more first antibodies and said one or more second antibodies each bind a prion protein associated with a prion disease or other related diseases.

35. The method of claim 34, wherein said prion disease is chronic wasting disease (CWD), bovine spongiform encephalopathy (BSE), kuru, Creutzfeldt-Jakob disease (CJD), variant Creutzfeldt-Jakob Disease (vCJD), Gerstmann-Straussler-Scheinker Syndrome, fatal familial insomnia, scrapie, transmissible mink encephalopathy, feline spongiform encephalopathy, or ungulate spongiform encephalopathy.

36. The method of claim 15, wherein said fluid sample is a biological material.

37. The method of claim 36, wherein said fluid sample further comprises water or a carrier.

38. The method of claim 37, wherein said fluid sample further comprises an aqueous buffer.

39. The method of claim 36, wherein said biological material is eyelid, blood, plasma, cerebrospinal fluid, neurological tissue, lymph, saliva, semen, feces, urine, aqueous humor, muscle, offal, or a combination, mixture, homogenate, extract, concentrate, or component thereof.
